(19) United States
(12) Patent Application Publication
(10) Pub. No.: US 2008/0026076 A1
(43) Pub. Date: Jan. 31, 2008

(54) Quercetin supplementation to treat hypertension

(76) Inventor: Thunder Jalili, Park City, UT (US)

Correspondence Address: NEEDLE & ROSENBERG, P.C., SUITE 1000, 999 PEACHTREE STREET, ATLANTA, GA, 30309-3915, US

(21) Appl. No.: 11/801,825

(22) Filed: May 11, 2007

Related U.S. Application Data
(63) Continuation-in-part of application No. 10/822,568, filed on Apr. 12, 2004.
(60) Provisional application No. 60/461,861, filed on Apr. 10, 2003.

(51) Int. Cl. A61K 31/352; A61K 31/355; A61K 31/4406; A61K 31/4415; A61K 31/714; A61K 33/04; A61K 33/06; A61K 33/26; A61K 33/30; A61K 33/34; A61P 9/12

(52) U.S. Cl. 424/638; 424/641; 424/646; 424/655; 424/682; 424/702; 514/345; 514/356; 514/456; 514/458; 514/52

(57) ABSTRACT

A nutritional supplement for improving cardiovascular health via aiding in preventing, slowing the progression of and/or treating hypertension, the supplement comprising quercetin; and a method for aiding in preventing, slowing the progression of and/or treating hypertension are described.

### CROSS-REFERENCE TO RELATED APPLICATION

This application is a continuation-in-part of U.S. patent application Ser. No. 10/822,568, filed on Apr. 12, 2004, which claims the benefit of U.S. Provisional Application No. 60/461,861, filed Apr. 10, 2003, the entire contents of each of which are incorporated by this reference.

### TECHNICAL FIELD

The invention relates to dietary supplements generally and more particularly to a method of using quercetin to treat hypertension.

### BACKGROUND

High blood pressure is known to cause abnormal growth and remodeling of the heart, known as cardiac hypertrophy. The stimulus for abnormal cardiac growth is an increase in cardiac workload due to high systemic blood pressure that must be overcome during ventricular contraction. Increased cardiac workload activates a number of biochemical pathways in the myocyte that trigger cardiac hypertrophy. Fifty million Americans currently suffer from high blood pressure and are undergoing some amount of cardiac hypertrophy and remodeling. Cardiac hypertrophy is a dangerous condition that increases the risk for arrhythmia, myocardial infarction, and heart failure. If left untreated, cardiac hypertrophy can progress at a gross and cellular level to the point where the cardiovascular architecture will not support normal function and will fail as a mechanical pump. This condition of heart failure is currently affecting 4.6 million Americans. In light of this situation, management of high blood pressure is critical to reducing the risk of cardiac hypertrophy and cardiac failure. A natural alternative that could decrease hypertension presents an opportunity to significantly reduce cardiac hypertrophy and promote cardiovascular health in the 50 million Americans currently suffering from hypertension.

Individuals suffering from hypertension have an increased risk of cardiac arrhythmia, cardiac hypertrophy, myocardial infarction, and heart failure. Chronic elevation of myocardial stress due to pressure overload, as in hypertension or aortic stenosis, causes cardiac muscle to undergo hypertrophy with a resulting increase in myocardial thickness. There is normally little or no change in overall cardiac size, so the wall thickening occurs at the expense of the cavity. One of the most damaging forms of hypertrophy is left ventricular hypertrophy (LVH). The prevalence of left ventricular hypertrophy in the population of borderline hypertensives has been estimated to be 16.6% (Melina et al., 1992). The data on prevalence are consistent, but not identical among studies. Some research which examined the role of hypertension and gender in the prevalence of LVH demonstrated rates of 14% and 20% for males and females in normotensives.

In the case of mitral regurgitation, on the other hand, ventricular volume increases with little or no change in maximum developed pressure; wall thickness does not alter significantly but total myocardial mass increases because of the ventricular enlargement. Except in the case of neonates, increase in mass does not alter the number of myocardial cells; the primary histological changes are intracellular, involving changes in the number and arrangement of the sarcomeres. In chronic cases widespread interstitial fibrosis occurs.

Accordingly, there is great interest in both dietary and pharmacological interventions that can prevent or treat existing hypertension. In addition, there is an enormous current public interest in alternative medicines and supplements. With regard to dietary intervention, consuming both flavonoid and vitamin antioxidants have great promise to prevent or reduce blood pressure in humans and experimental animals (Brody et al., 2002; Aviram et al., 2001; Kobaet al., 1992; and Duarte et al., 2001). Compared to pharmacological agents, dietary supplements can be less expensive and have greater popular appeal. Since the use of alternative medicines has greatly increased among Americans in recent years, flavonoid based anti-hypertensive supplements have great commercial potential.

In recent years there has been strong interest in the role phytonutrients may play in preventing heart disease. Dietary flavonoids have received particular attention since many are strong antioxidants and have been shown to reduce the risk of cardiovascular diseases by reducing blood pressure and slowing atherosclerosis.

Recent studies have demonstrated in animals and humans that antioxidants can decrease blood pressure. Furthermore, a number of in vitro studies have demonstrated that quercetin can inhibit Protein Kinase C (PKC), a family of biochemical signaling molecules implicated in governing cardiac hypertrophy and failure.

What is needed therefore is a natural alternative that could decrease blood pressure, which may simultaneously act upon the biochemical pathways that govern cardiac hypertrophy, to significantly reduce cardiac hypertrophy and remodeling in the 50 million Americans currently suffering from hypertension.

### SUMMARY OF THE INVENTION

The invention provides a nutritional supplement composition comprising quercetin. More particularly it provides a nutritional supplement formulation containing a prophylactically effective amount of quercetin that is specifically dedicated to ameliorating, delaying and/or treating hypertension.

The present invention also provides a method for ameliorating, delaying and/or treating hypertension, which comprises administering the nutritional supplement composition to an individual who is at risk or suffers from hypertension.

### BRIEF DESCRIPTION OF THE DRAWINGS

FIG. 1 illustrates mean carotid arterial pressure in groups of rats fed either control diet or a diet supplemented with quercetin and either having had a sham surgery or abdominal aorta constriction (AAC).

FIG. 2A illustrates the pressure overload produced with abdominal aortic constriction (AAC) surgery in rats as compared to rats having a sham operation (SH). PKC βII translocates to the cellular membrane fraction in the heart and this translocation of PKC βII has been previously identified as a critical mediator of cardiac hypertrophy. FIG. 2A also illustrates that quercetin supplementation prevents cardiac PKC βII translocation, and attenuates cardiac hypertrophy. FIG. 2B demonstrates that the other PKC isoforms in the heart are unchanged by both AAC and quercetin supplementation. AAC=abdominal aortic constriction, SH=sham operated, AACQ=abdominal aortic constriction+quercetin, SHQ=sham operated+quercetin.

FIG. 3 illustrates that quercetin prevents vascular dysfunction. AAC rats typically have impaired endothelial dependent relaxation in their aortas when stimulated with acetylcholine (which stimulates vasodilation). Rats given supplemental quercetin have normal endothelial dependent relaxation under the same acetylcholine dose. AAC=abdominal aortic constriction, SH=sham operated, AACQ=abdominal aortic constriction+quercetin, SHQ=sham operated+quercetin. * p<0.05.

FIGS. 4A & 4B illustrates that activation of Akt is prevented and ERK1/2 is reduced in rats with abdominal aortic constriction fed supplemental quercetin. It has been previously demonstrated that activation of Akt and ERK1/2results in cardiac hypertrophy. AAC=abdominal aortic constriction, SH=sham operated, AACQ=abdominal aortic constriction+quercetin, SHQ=sham operated+quercetin. *, ** p<0.05.

FIGS. 5A, 5B, and 5C illustrate a quercetin dependent delay in the onset of hypertension using a genetic model of hypertension. Spontaneously hypertensive rats suffer from steadily increasing blood pressure from 6 weeks of age until peaking at 12-15 weeks of age. SH rat diets were supplemented with 1.5 g/kg chow quercetin (SHRQ), which delayed the normal rise in blood pressure. FIG. 5A illustrates a decreased systolic blood pressure in SH rats fed a diet supplemented with quercetin. FIGS. 5B and 5C illustrate that the diastolic pressure and mean arterial pressure did not show a statistically significant changed. *p<0.05.

FIG. 6 illustrates a summary of subject recruitment and experimental design.

FIG. 7 illustrates change in blood pressure from baseline in pre-hypertensive (n=22) and stage 1 hypertensive subjects (n=22) subjects who participated in a placebo-controlled, crossover-design, double-blind, clinical trial to test the efficacy of 730 mg quercetin/day to reduce blood pressure. Data are means±SEM. MAP, mean arterial pressure.

### DETAILED DESCRIPTION OF THE INVENTION

The consumption of quercetin is quite low in the United States today, estimated at 25 mg/day. This is due to inadequate consumption of quercetin containing foods, in particular, onion (one of the best source of quercetin) consumption is lower in the United States as compared to other parts of the world.

The invention provides a nutritional supplement composition comprising quercetin. More particularly it provides a nutritional supplement formulation containing a prophylactically effective amount of quercetin that is specifically dedicated to ameliorating, delaying and/or treating hypertension.

The present invention also provides a method for ameliorating, delaying and/or treating hypertension, which comprises administering the nutritional supplement composition to an individual who is at risk of or suffers from hypertension.

Embodiments of the invention include determining if a subject is suffering from hypertension or is prone to the development of hypertension. Determining if the subject is prone to hypertension may include determining if the subject is suffering from prehypertension. Determining if the subject is suffering from hypertension or prehypertension may include measuring the subject's blood pressure. Blood pressure may be measured a variety of ways, such as with an sphygmomanometer (also referred to as an sphygmometer) or Omron random zero blood pressure analyzer. Measuring blood pressure may include multiple measurements over the course of several weeks and/or multiple measurements in a single day. Determining if a subject is suffering from hypertension may first include evaluating whether the subject has symptoms of hypertension. Symptoms of hypertension may include headaches, blurred vision, dizziness, ringing in one or both ears, and mental confusion. In some cases, a subject may have hypertension and not have any symptoms.

Embodiments of the invention include a method of treating a subject. The method includes measuring the subject's blood pressure, and then comparing the measured blood pressure with a normal blood pressure for such a subject. If the measured blood pressure exceeds the normal blood pressure for such a subject, then diagnosing (i.e., determining) if the subject is suffering from or is at risk from suffering from hypertension. If the subject is diagnosed as suffering from or at risk from suffering from hypertension, then directing the subject to ingest a nutritional supplement comprising quercetin, regularly, in amounts sufficient to treat hypertension or prehypertension in the subject. The method includes continuing to measure the subject's blood pressure at regular intervals to measure the efficacy of the ingestion of the nutritional supplement to treat the hypertension or reduce the risk of the subject suffering from hypertension.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed, and vice versa. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

As used herein “a subject” means an animal, including, but not limited to, a human and other mammals.

In this specification and in the claims which follow, reference will be made to a number of terms which shall be defined to have the following meanings: “Treating” or “treatment” does not mean a complete cure. It means that the symptoms of the underlying disease are reduced, and/or that one or more of the underlying cellular, physiological, or biochemical causes or mechanisms causing the symptoms are reduced or delayed. It is understood that reduced or delayed, as used in this context, means relative to the state of the disease, including the molecular state of the disease, not just the physiological state of the disease.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions.

Thus, if there are a variety of additional steps that can be performed it is understood that each of these additional steps can be performed with any specific embodiment or combination of embodiments of the disclosed methods.

As used herein an “effective amount” means a quantity of quercetin and quercetin glycosides, wherein an average adult would experience the health benefits of the compound(s). The health benefits include the prevention, delay and/or treatment of hypertension and the promotion of cardiovascular health. In addition, a dose may be administered over the course of multiple time periods, such as being administered twice daily (one half the daily dose per administration) or once a day. For example, an effective dosage for humans includes a dosage of about 50 mg/kg body weight, which would translate to about 3750 mg per day for a 75 kg human. An effective amount of quercetin includes between about 100 mg/day (0.1 g/day) and about 50000 mg/day (50 g/day), preferably between about 1000 mg/day and 30000 mg/day, more preferably between about 1000 mg/day and about 15000 mg/day, yet more preferably 1000 mg/day and about 5000 mg/day. In an exemplary embodiment, quercetin is provided to a human subject in a dosage of between 100 mg and 2000 mg/day. Dosage is related to the body mass, health status, age and the desired effect relative to an individual. Therefore, the dosage may be varied according to the administration schedule, body mass, age or the like. The dosages set forth herein are safe even for an adult of low body mass, e.g. a 100 pound adult. No toxic effects at the highest dosage set forth herein are known. However, the dosages set forth herein are preferably administered at the lower dosages for subjects having a smaller body weight and at higher dosages for subjects having a larger body weight.

As used herein “hypertension” means an elevation of arterial blood pressure over the accepted norms for a given age (either primary or secondary) and disease caused by elevated blood pressure, including, left ventricular hypertrophy (LVH), right ventricular hypertrophy (RVH), Biventricular Hypertrophy, ischemia of the myocardium and myocardial infarction. Hypertension is defined as systolic blood pressure of 140 mm Hg and above, diastolic blood pressure of 90 or above, or both. The American Heart Association defines pre-hypertension as systolic blood pressure of 120-139, diastolic pressure of 80-89, or both.

Flavonoids, also known as “phenylchromones,” are naturally occurring, water-soluble compounds which have antioxidant characteristics. Flavonoids are found in a variety of vascular plants, such as vegetables and fruits. As a result, flavonoids are present in beverages such as tea and wine (particularly red wine). In contrast to vascular plants, members of the animal kingdom are unable to synthesize the flavone nucleus and obtain flavonoids as a dietary component. Tea and wine, particularly, red wine, may be used to reduce hypertension by the methods of the invention.

It is believed that quercetin, which exhibits some of the strongest antioxidant effects of the flavonoids and reportedly inhibits oxidation and cyto-toxicity of low density lipoproteins (LDL), may have beneficial health consequences since oxidized low density lipoproteins are reported to contribute to the buildup of fatty substances in the arterial wall (to be atherogenic). Lipid peroxidation, which is caused by free radicals (highly reactive molecules with at least one unpaired electron) can be reduced by the antioxidant activity of flavonoids and quercetin.

Flavonoids are conjugated aromatic compounds having the general structure: 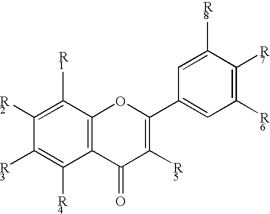
where R₁, R₂, R₃, R₄, R₅, R₆, R₇ and R₈ are independently selected from H and OR′ where R′ is H or an alkyl group having about 1 to 10 carbon atoms (see also, U.S. Pat. No. 6,203,818, incorporated by reference). The most widely occurring flavonoids are flavones and flavonols. The present invention contemplates the use of all flavonoids, however, flavonols and more particularly, quercetin (3,5,7,3′,4′-pentahydroxyflavone), myricetin, (3,5,7,3′,4′,5′,-hexahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof are preferred. A preferred flavonoid for use in the invention is quercetin and quercetin glycosides, which are used to illustrate the invention.

Quercetin (3,5,7,3′,4′-pentahydroxyflavone) and quercetin-4-glycoside (hereinafter referred to as quercetin) (Graefe et al., 2001) prevents and/or treats hypertension and reduces blood pressure in established hypertension. Quercetin is a naturally occurring antioxidant found in highest concentrations particularly in onions. Quercetin is also naturally occurring in apples and berries. It is classified as a flavenoid characterized by 2 benzene rings linked through a heterocyclipyrone ring. It has been reported that quercetin is an antioxidant 10 times more potent than vitamin C (Pedro-Botet et al., 2000).
Structure of Quercetin:

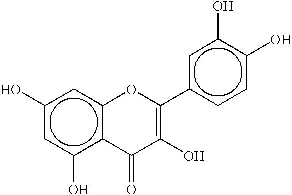

Quercetin acts as an antioxidant that is believed to directly reduce systemic blood pressure and is further believed to act upon the key biochemical pathways responsible for cardiac growth induced by high blood pressure. In one embodiment, a quercetin supplement is prepared as nutritional food, such as a food bar, energy bar, performance snack or performance gel used in preventing and/or treating hypertension. The products produced by POWERBAR® provide non-limiting examples of energy bars, performance snacks and/or energy gels (see also, U.S. Pat. No. 6,248,375, hereby incorporated by reference). In another embodiment, a quercetin supplement is prepared as a liquid beverage, smoothie, diet shake, meal substitute shake, low carbohydrate drink, or health drink. For example, a non-limiting example is a smoothie having quercetin, such as a Jamba Juice® booster. In another embodiment, quercetin is added to a beverage, such as orange juice. In yet another embodiment of the invention a dietary fiber supplement such as oat bran or other natural fiber source may also be added to the composition. In another exemplary embodiment, other supplements such as fish oil or other neutraceutical supplements may be added. A daily supplement containing quercetin may be effective in preventing hypertension in over 50 million people who are at risk for hypertension. With regard to patients with established hypertension, quercetin, a natural agent, may be used to treat this disease. In another exemplary embodiment, a daily supplement containing quercetin may be used to delay the on-set of the hypertension.

The model of using enriched food products to prevent cardiovascular disease currently exists and is economically successful. Three separate margarine products are currently being sold (Benecol®, Take Control®, Smart Balance®). These products have artificially enriched levels of phytosterols, which have been shown to reduce blood cholesterol levels in animal models and clinical studies. Accordingly, these products have been marketed as agents to reduce risk of myocardial infarction.

Hypertensive humans have decreased antioxidant capacity, presumably due to the oxidative stress taxing the body's antioxidant systems (Pedro-Botet et al., 2000). It has been demonstrated in rats that depletion of natural antioxidant systems produced hypertension (Vaziri et al., 2000a; Vaziri et al., 1999). In addition, antioxidant supplementation in hypertensive animal models has been shown to be effective in reducing blood pressure in both spontaneously hypertensive rats, and in rats depleted of their natural antioxidant systems (Newaz and Newal, 1999). Human subjects taking both an anti-hypertensive medication and antioxidant supplements (e.g., Zinc, vitamin E, etc.) have a greater reduction in blood pressure than those on medication alone (Galley et al., 1997). It is believed that antioxidants sequester reactive oxygen species and preserve the levels of nitric oxide, a powerful vasodilator (Vaziri et al., 2000b). Quercetin is believed to be effective in reducing blood pressure in hypertensive models due to its strong antioxidant capacity, documented to be 10 times more potent than vitamin C (Naguib, 2000; Pedro-Botet et al., 2000).

From a variety of models and species, the involvement of PKC in hypertrophy has been demonstrated. Protein Kinase C (PKC) is one of the key signaling pathways implicated in the pathogenesis of cardiac hypertrophy and heart failure. PKC is an enzyme family of serine-threonine kinases in mammalian cells and is thought to activate downstream signaling pathways and genes governing cardiac cell growth. PKC in the heart has been shown to be activated by high blood pressure and to play a role in cardiac hypertrophy in cell culture, conventional animal models, genetically engineered mice, and humans. Mechanical stretch of cardiac myocytes in culture (mimicking hypertension) activates PKC, turns on hypertrophic genes and leads to myocyte hypertrophy. Experimentally induced high blood pressure in rats and guinea pigs also activates PKC and leads to cardiac hypertrophy (Jalili, et al., 1999; Gu and Bishop, 1994). In transgenic mice, cardiac specific overexpression of PKC βII isoform results in cardiac hypertrophy and 100% mortality (Wakasaki et al., 1997). Most importantly, humans in heart failure also have increased PKC activation (Bowling et al., 1999).

Quercetin has been identified as a PKC inhibitor in cell culture. Previous studies have found inhibition of PKC activity in the cytosol and membrane fraction of cells isolated from thyroid (Picq et al., 1989), brain (Ferriola et al., 1989), liver (Mistry et al. 1997), and fibroblasts (Lee and Lin, 1997). These studies all used similar protocols; incubation cells with similar (i.e., μM) concentrations of quercetin. However, there is very little in vivo data examining the effect of quercetin on PKC activity.

There has been one study examining the role of quercetin to prevent cardiac hypertrophy in mice (Wang et al., 1999). An oral, 5 day, 120 mg/kg/day dose of quercetin prevented cardiac hypertrophy in mice with surgically induced abdominal aortic constriction designed to produce pressure overload. The mechanism for this observation has not been identified.

Quercetin, or quercetin glycosides or similar isoforms can serve as an economical natural agent to fight hypertension, and may be combined with one or more other products to further lower the risks of cardiovascular disease. For example, quercetin may be added to well known nutritional supplements and/or non-flavonoid antioxidants, e.g., selenium, vitamin E (tocopherol, particularly α- and δ-tocopherol, etc.), vitamin C (ascorbic acid) and coenzyme Q10 and/or dietary fiber supplements. Quercetin may be added to other flavonoids and flavonols, such as myricetin, (3,5,7,3′,4′,5′,-hexahydroxyflavone), kaempferol (3,5,7,4′-tetrahydroxyflavone), and flavones apigenin (5,7,4′-trihydroxyflavone) and luteolin (5,7,3′,4′-tetrahydroxyflavone) and glycosides thereof.

In addition to quercetin, the supplement of the invention may also contain a vitamin B complex member, such as vitamin B₁₂ and vitamin B₆. Each member of the vitamin B complex group is a distinctly different substance with different functions. Therefore, quercetin may be combined with one or more vitamin B complex member.

Other well known nutritional supplements such as amino acids and derivatives thereof, e.g., L-arginine, non-flavonoid antioxidants, e.g., selenium, vitamin E isoforms (α- or δ-tocopherol, etc.), vitamin C (ascorbic acid), coenzyme Q10, niacin and beta-carotene may be effectively used in the nutritional supplement of this invention.

Other additives may be incorporated in the nutritional supplement of the present invention. Such additives include minerals, e.g., boron, etc. and trace metals such as zinc, magnesium, manganese, chromium, molybdenum, copper, iron, calcium, and potassium; and other micronutrients such as thiamine, riboflavin, niacin, pantothenic acid, pyridoxine, choline, biotin, inositol, para-aminobenzoic acid, vitamin D, vitamin K, vitamin A, etc. In another embodiment of the invention a dietary fiber supplement such as oat bran or other natural fiber source may also be added to the composition. In one embodiment, quercetin is formulated to produce a food or food bar. In another embodiment, quercetin is added to an aqueous based drink.

The nutritional supplement may, where desirable and appropriate, further include a pharmaceutically acceptable carrier such as lactose, glucose, sucrose, corn starch, potato starch, cellulose acetate, ethyl cellulose, etc. Diluents and other additives such as one or more pharmaceutically acceptable binding agents, fillers, supports, thickening agents, taste-improving agents, coloring agents, preservatives, stabilizers, regulators, emulsifiers or mixtures thereof may be used depending on the form of the composition employed.

The supplement is preferably administered orally but may be administered parenterally or topically. Suitable forms for oral, topical or parenteral administration include tablets, capsules, lozenges, syrups, granules, solutions, sub-dermal patch, lotion, and suspensions, which contain unit doses of the supplement for administration once or several times a day; or at various time intervals. The nutritional supplement composition of the invention will typically be administered orally as a tablet, capsule, gel tabs, or liquid. More preferably, the nutritional supplement composition of the invention will typically be administered orally as a food or energy bar or as a nutritional drink. In addition, sustained release formulations can be formulated and prepared according to manufacturing techniques well known in the pharmaceutical industry and in a variety of dosage forms.

The examples demonstrate the use of quercetin to improve blood pressure and vascular function, thereby, attenuating cardiac hypertrophy and contractile and/or vascular dysfunction.

### EXAMPLE I

To model pressure overload, a surgically induced procedure was used to mimic high blood pressure, known to lead to cardiac hypertrophy. In this procedure the abdominal aorta is constricted to increase the afterload on the heart and produce pressure overload. Male Sprague Dawely 225-250 g rats (n=10) were placed on a diet consisting of 0.15% purified quercetin. Purified quercetin (purchased from Sigma Aldrich) was mixed into standard rat chow at a concentration of 0.15% (1.5 mg quercetin/g of chow). Rats consumed this diet for 7 days.

Given that a rat generally consumes ten grams of chow per 100 g body weight when given free access to the chow, a 250 g rat consumes about 37.5 mg quercetin per day under this regimen. Expressed in pharmacological terms, this is equivalent to a dose of 150 mg quercetin/kg body weight/day.

On the 8th day, 5 rats were surgically modeled for pressure overload by constricting the abdominal aorta to a diameter of 0.63 mm with a Weck hemoclip. The other 5 rats were sham operated (anesthetized and the aorta exposed, but not constricted). The procedure of aortic constriction mimics hypertension, is reproducible, and has been demonstrated in previous studies to induce cardiac hypertrophy after only 2 weeks. Another group of 10 rats were placed on a standard diet with no supplement and treated in an identical fashion with 5 surgically modeled for pressure overload and 5 sham operated. Thus 4 experimental groups were used in this protocol; Sham operated (S) n=5, abdominal aorta constricted (AAC) n=5, Sham operated+quercetin (SQ) n=5, and abdominal aorta constricted+quercetin (AAC-Q) n=5. Two weeks after surgery post-sacrifice morphometric measurements are obtained by measuring total heart weight, left ventricle weight, and calculating heart:body weight and left ventricle:body weight ratios to assess cardiac mass and cardiac hypertrophy. It is found that AAC rats develop significant cardiac hypertrophy compared to sham operated controls. In comparison, AAC-Q rats that were fed a diet supplemented with quercetin have a lower level of cardiac hypertrophy.

In ongoing experiments with greater numbers of rats, it was found that quercetin treated rats (AAC-Q) have levels of hypertrophy in between S and AAC rats.

### EXAMPLE II

An identical experimental procedure as described in Example I is used with n=10 rats per group (S, AAC, SQ, AAC-Q) for a total of 40 rats. Surgically induced AAC is again the model for the development of pressure overload and cardiac hypertrophy. Rats subjected to this protocol have very high carotid blood pressure. Rats are anesthetized, a catheter is placed in the caudal and carotid arteries, rats are allowed to regain consciousness for 60 minutes and blood pressures are measured.

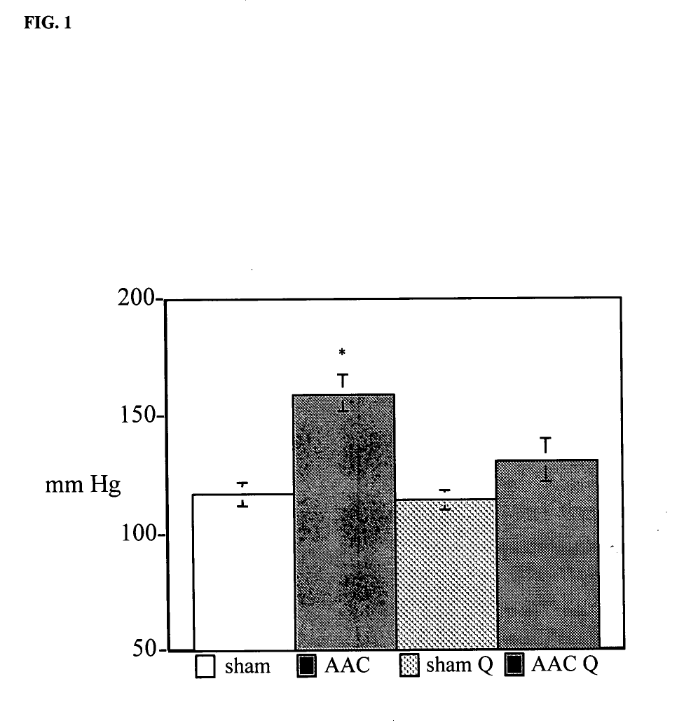

As illustrated in FIG. 1, Quercetin, a dietary flavenoid, reduces blood pressure in the carotid arterial blood pressure in the AAC-Q group. Rats in the AAC-Q group also have attenuated cardiac hypertrophy compared to untreated AAC rats. Therefore, the in vivo data generated with pressure overloaded rats provides information regarding the beneficial effects of quercetin on blood pressure in human subjects.

Mean carotid arterial pressure in AAC rats is approximately 37% greater than controls, however, when AAC rats are supplemented with quercetin (1.5 g/kg of chow), mean carotid arterial pressure is normalized (FIG. 1). Thus, quercetin reduces carotid arterial pressure and consequently reduces the stimulus (pressure overload) for cardiac hypertrophy.

Systolic carotid pressures are significantly reduced in AAC-Q rats compared to AAC (Table 1) (158.8±10.3 vs 201.4±11.4). Diastolic carotid pressures in AAC-Q rats are normalized to the same level of S and SQ (Table 1).

Both systolic and diastolic pressure gradients between caudal and carotid arteries are reduced in AAC-Q rats compared to AAC. Due to the physical constriction of the abdominal aorta, there is a greater pressure in the carotid artery compared to the caudal artery. Thus a pressure gradient exists in AAC rats. AAC-Q rats have reduced systolic pressure gradients and normal diastolic pressure gradients (see Table 1).

Taken together, these data indicate that quercetin supplementation can reduce and/or prevent increases in blood pressure produced by AAC.

TABLE 1
Blood pressure measurements (mmHg) in caudal and carotid arteries of
conscious rats¹.

| | Sham | AAC | Sham + Q | AAC + Q | Significance |
|---|---|---|---|---|---|
| Systolic Caudal | 121.2 ± 2.6 | 125.0 ± 5.8 | 114.7 ± 3.2 | 118.3 ± 6.4 | NS |
| Systolic Carotid | 131.1 ± 4.1^{ab} | 201.4 ± 11.4^{c} | 128.2 ± 1.9^{a} | 158.8 ± 10.3^{b} | <0.001 |
| Systolic Gradient | 4.6 ± 2.8^{a} | 71.2 ± 9.6^{c} | 7.3 ± 1.9^{a} | 38.6 ± 5.7^{b} | <0.001 |
| Diastolic Caudal | 105.4 ± 5.5^{b} | 113.7 ± 5.5^{b} | 93.6 ± 3.2^{a} | 104.7 ± 5.5^{b} | 0.049 |
| Diastolic Carotid | 109.9 ± 5.2^{a} | 139.6 ± 7.2^{b} | 106.2 ± 4.8^{a} | 118.4 ± 6.1^{a} | 0.007 |
| Diastolic Gradient | 4.5 ± 2.8^{a} | 22.0 ± 3.1^{b} | 7.0 ± 2.9^{a} | 8.8 ± 2.6^{a} | 0.002 |
| Caudal MAP² | 108.7 ± 2.3 | 117.8 ± 5.6 | 100.6 ± 3.0 | 108.0 ± 7.1 | NS |
| Carotid MAP² | 117.2 ± 4.6^{a} | 160.1 ± 7.9^{b} | 113.5 ± 3.8^{a} | 130.9 ± 9.2^{a} | 0.001 |
| Gradient MAP² | 4.6 ± 2.2^{a} | 37.9 ± 4.7^{c} | 7.2 ± 1.9^{ab} | 18.3 ± 4.4^{b} | <0.001 |

¹Values are means ± standard error of mean. Different letters indicate significant differences at P < 0.05
²MAP = mean arterial pressure.
Sham = sham operated rats,
AAC = rats with abdominal aortic constriction,
Sham + Q = sham operated rats treated with quercetin,
AAC + Q = rats with abdominal aortic constriction treated with quercetin.
N = 5 to 10 rats/group.

### EXAMPLE III

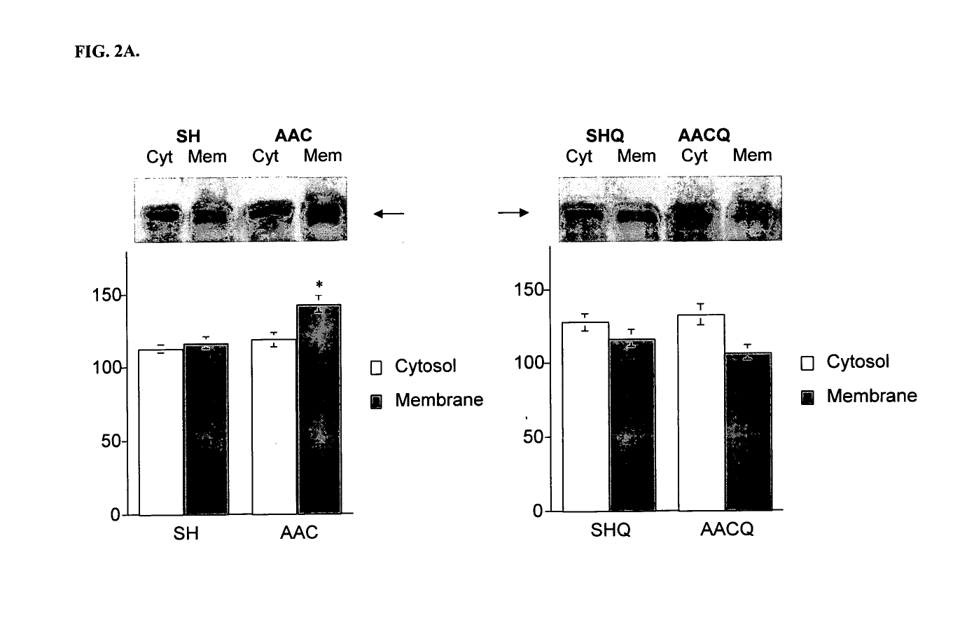

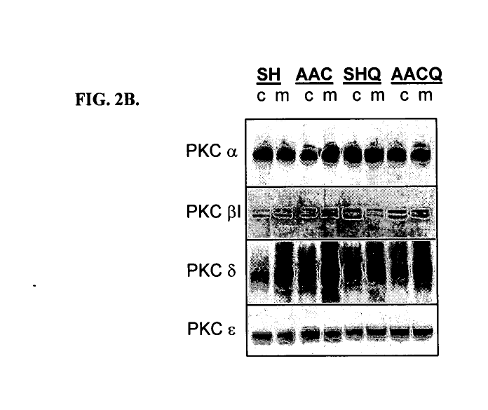

An identical experimental protocol as described in Example I and II was performed. PKC α, βI, βII, ε, δ levels were characterized in each group, as shown in FIGS. 2A & 2B. Following the experimental period, rats were sacrificed, left ventricles were homogenized, and cytosolic & membrane cell fractions isolated using differential centrifugation as previously described (Jalili et al., 1999). Immunobloting using standard protocols was used to assess PKC distribution. Id. Increased abundance in membrane bound fractions is indicative of PKC activation.

Activation of PKC isoforms assessed using Western blots indicated no significant differences in α, βI, ε, or δ isoforms between all groups (see, FIG. 2B). PKC βII translocation and expression was upregulated in AAC and significantly reduced in AACQ. In particular, quercetin supplementation reduced translocation of PKC βII to the membrane fraction (compare AAC with AACQ, as shown in FIG. 2A). Translocation has been identified as a critical mediator of cardiac hypertrophy. Therefore, dietary quercetin may have reduced cardiac hypertrophy during pressure overload and attenuate cardiac hypertrophy. Without wishing to be bound by a theory, the reduction in cardiac hypertrophy may involve the inhibition of PKC βII brought about independently by quercetin supplementation or resulting indirectly from a reduction in blood pressure described in Example II.

PKC blots may be quantified using a UMAX Astra 1220U Scanner, Adobe Photoshop (V 5.0) and NIH Image software (V 1.61). Band density may be measured as scanning pixel units and statistical analyzed using programs such as Sigma Stat software (V 2.01).

### EXAMPLE IV

Using the experimental protocols described in the previous examples with n=7 to 10 per group, the effects of quercetin on vascular reactivity is assessed. Animals are anesthetized, the heart and aorta excised, and vascular reactivity is assessed in segments of aorta using a wire-type myograph.

Post-sacrifice morphometric measurements are obtained by measuring body weight, total heart weight, calculating heart:body weight ratios and heart rate to assess cardiac mass and cardiac hypertrophy. Administration of quercetin reduces cardiac hypertrophy (see, Table 1) and vascular dysfunction associated with cardiac hypertrophy, as demonstrated in experiments described herein.

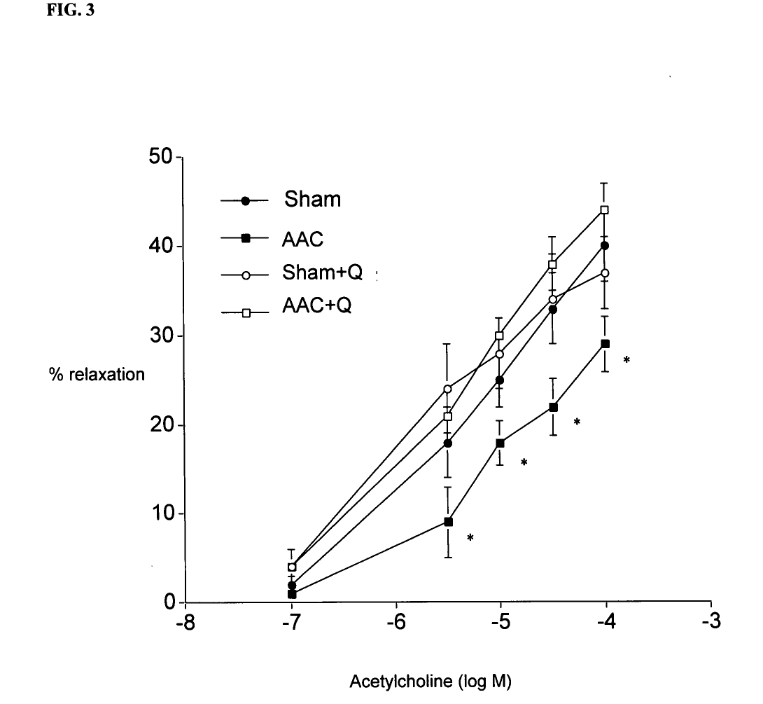

Supplementing quercetin in the diet prevents aortic dysfunction associated with pressure overload and cardiac hypertrophy. AAC-Q rats demonstrate greater endothelium-dependent relaxation in isolated aorta. Therefore, chronic in vivo quercetin-supplementation prevents aortic vascular dysfunction associated with pressure overload in the AAC rat (see, FIG. 3).

As shown in Table 2 and FIG. 3, compared to sham-operated control fed animals (n=10), AAC rats (n=10) had a greater heart weight/body weight ratio (2.93±0.04 vs 3.76±0.18* mg/g), less maximal ACh-evoked relaxation (10⁻⁴ M, 40±4 vs 30±3%*), and less SNP-evoked relaxation (10⁻⁴ M, 71±5 vs 56±3%*) in NE precontracted (10⁻⁵M) aortic segments.

Compared to sham-operated Q-fed animals (n=10), AAC-Q rats (n=9) had a greater heart weight/body weight ratio (2.97±0.06 vs 3.46±0.06*), but similar maximal ACh-evoked relaxation (38±4 vs 44±3%) and SNP-evoked relaxation (71±5 vs 71±4%) in NE precontracted aortic segments (*p<0.05 for all).

Compared to AAC, AAC-Q rats had improved Ach-evoked relaxation (44±3 vs 30±3%*) and improved SNP-evoked relaxation (71±4 vs 56±3%*) (*p<0.05 for all). These data demonstrate that quercetin supplementation reduces blood pressure and prevents aortic dysfunction typically associated with AAC.

TABLE 2
Body weight, organ weights, and levels of quercetin in plasma and liver¹

| | Sham | AAC | Sham + Q | AAC + Q | Significance |
|---|---|---|---|---|---|
| Body wt. (g) | 335 ± 7 | 321 ± 6 | 342 ± 10 | 342 ± 7 | NS |
| Heart wt. (g) | 0.98 ± 0.02^{a} | 1.24 ± 0.06^{b} | 1.01 ± 0.03^{a} | 1.18 ± 0.03^{b} | <0.001 |
| Heart (mg): body wt (g) | 2.93 ± 0.04^{a} | 3.76 ± 0.18^{c} | 2.97 ± 0.06^{a} | 3.46 ± 0.06^{b} | <0.001 |
| Heart Rate (BPM) | 382 ± 16^{a} | 410 ± 8^{b} | 402 ± 9^{b} | 414 ± 9^{b} | 0.002 |
| Lung wt. (g) | 1.46 ± 0.05 | 1.45 ± 0.07 | 1.50 ± 0.05 | 1.48 ± 0.05 | NS |
| Liver wt. (g) | 12.3 ± 0.5 | 12.1 ± 0.5 | 11.1 ± 0.7 | 10.6 ± 0.6 | NS |
| Plasma quercetin (μg/mL)² | 0^{a} | 0^{a} | 3.26 ± 0.11^{b} | 3.96 ± 0.89^{b} | <0.001 |
| Liver quercetin (μg/mL)³ | 0^{a} | 0^{a} | 3.00 ± 0.47^{b} | 3.52 ± 0.89^{b} | <0.001 |

¹Values are means ± standard error of mean. Different letters indicate significant differences at P < 0.05
²Plasma quercetin levels composed of free and conjugated quercetin.
³Liver quercetin levels composed of free quercetin, free and conjugated O-methoxy quercetin
Sham = sham operated rats (n = 10),
AAC = rats with abdominal aortic constriction (n = 10),
Sham + Q = sham operated rats treated with quercetin (n = 10),
AAC + Q = rats with abdominal aortic constriction treated with quercetin (n = 9).

Left Ventricular Hypertrophy (LVH) in a subject may be diagnosed using echocardiography (ECG) techniques (Am Heart J, 1949;37:161; Circulation, 1987;3: 565-72; Circulation,1990; 81:815-820; and Am Heart J, 1986:75:752-58). A subject meeting the criteria is highly likely to suffer from LVH. The general ECG criteria used to diagnose LVH include, a increased QRS amplitude (voltage criteria; i.e., tall R-waves in LV leads, deep S-waves in RV leads), delayed intrinsicoid deflection in V6 (i.e., time from QRS onset to peak R is ≧0.05 sec), widened QRS/T angle (i.e., left ventricular strain pattern, or ST-T oriented opposite to QRS direction), and a leftward shift in the frontal plane QRS axis (Lipman B, Cascio T. ECG Assessment and Interpretation. Philadelphia, Pa.:FA Davis Co.; 1994).

In this model of LVH, Q-supplementation limits aortic vascular dysfunction. In addition, quercetin treatment significantly reduces, blood pressure, PKC βII activation, and cardiac hypertrophy.

### EXAMPLE V

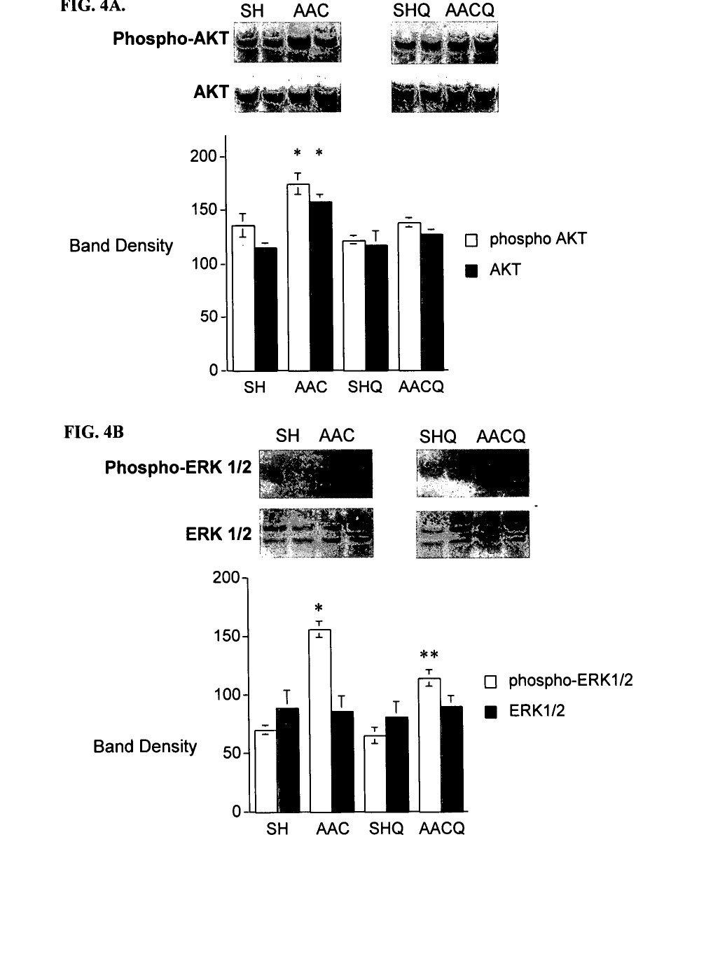

Determination of ERK1/2 and Akt signaling status (FIGS. 4A & 4B). All extraction procedures were performed at 4° C. Left ventricles from Sham (n=5), AAC (n=5), Sham+quercetin (n=5), and AAC+quercetin (n=5) were homogenized with a tissuemizer in 1 ml of ice-cold RIPA buffer (50 mM Tris-HCl (pH 7.4), 150 mM NaCl, 1% Nonidet P-40, 0.25% sodium deoxycholate, 1 mM sodium orthovanadate, 1 mM NaF, and 10 μl/mL Sigma protease inhibitor cocktail (Sigma, St. Louis, Mo., cat. #P-8340)). After homogenization, the samples were sonicated twice on ice and centrifuged at 11,000×g for 10 min. at 4° C. Supernatants were recovered and stored at −80° C. for subsequent immunoblotting. Protein concentration of whole heart cell lysate was determined using a Bio-Rad Protein assay (Bio-Rad, Hercules, Calif.) with bovine serum albumin (BSA) as a standard.

Western blotting, transfer and densitometry: Electrophoresis and transfer of proteins to PVDF membranes were done as previously described (18, 19). Primary antibody directed against PKC α, βI, βII, δ, and ε (Santa Cruz Biotechnology, Santa Cruz, Calif.) were incubated overnight at 4° C. in a 1:1000 dilution. Antibodies directed against phospho ERK 1/2, phospho JNK, ph c-Raf (Cell Signal Technology, Beverly, Mass.), and phospho PKC P (Santa Cruz Biotechnology, Santa Cruz, Calif.), were incubated at a 1:1000 dilution for 48 h at 4° C. in 5% BSA Tris buffer with 0.05% Tween-20. Secondary antibody conjugated to horseradish peroxidase (goat anti rabbit, (Cell Signal Technology, Beverly, Mass.)) was incubated for 1 h at 1:10,000 dilution. Signals were visualized by enhanced chemiluminescence (Cell Signal Technology, Beverly, Mass.). Relative band density of immunoblots on film were measured with a scanner using NIH 1.63 image software (National Institutes of Health, Rockville, Md.).

### EXAMPLE VI

The spontaneously hypertensive (SH) rat is used to demonstrate the effectiveness of quercetin, since this strain is well documented to have normal blood pressure until 5-6 weeks of age (Makita and Yasuda, 1990). After 5-6 weeks, blood pressure steadily rises until reaching a peak systolic pressure of 200-220 mm Hg at 11 weeks of age. This etiology is similar to humans predisposed to hypertension that exhibit rising blood pressure with age. As a result of developing hypertension, SH rat also develops cardiac hypertrophy by 8 weeks of age (Rizzoni et al., 1994). Therefore, the SH rat has been recognized as an ideal model to test suitability of blood pressure lowering agents (Roba, 1976).

Data regarding the effectiveness of quercetin in the rat studies may be analyzed using a one way ANOVA (SPSS v. 10) with LSD post hoc test used to detect significant differences between groups.

Male weanling WKY and SH rats (4 weeks of age) (purchased from Harlan, Indianapolis, Ind.) are measured for baseline blood pressures and body weights and randomly divided into dietary groups; Group 1; WKY normotensive+control diet, Group 2; SH rat+control diet, Group 3; SH rat+1.5 g quercetin/kg diet. After 5 and 10 weeks of diet treatment blood pressure, echocardiograms and body weight are measured. Animals are sacrificed after the 10^{th} week of diet treatment and coronary arteries are removed to determine endothelial function and nitric oxide production.

### EXAMPLE VII

The SH rat (SHR) was used to demonstrate the effectiveness of quercetin (20). As described herein, this strain is well documented to have normal blood pressure until about 5-6 weeks of age (Makita and Yasuda, 1990). After about 5-6 weeks, the rats suffer from steadily increasing blood pressure from about 6 weeks of age until peaking at about 12-15 weeks of age.

Blood pressure was measured in SHR and SHRQ rats by lightly anesthetizing the animals using ketamine/xylazine (75/3.75 mg/kg i.p.). Rats were prewarmed for 15 minutes on heated platform and blood pressure was measured using a Visitech BP 2000 blood pressure system. Pressure measurements are reported as the average of between 4 and 8 separate measurements taken over a 70 second period. All rats fully recovered within 30 after receiving anesthesia.

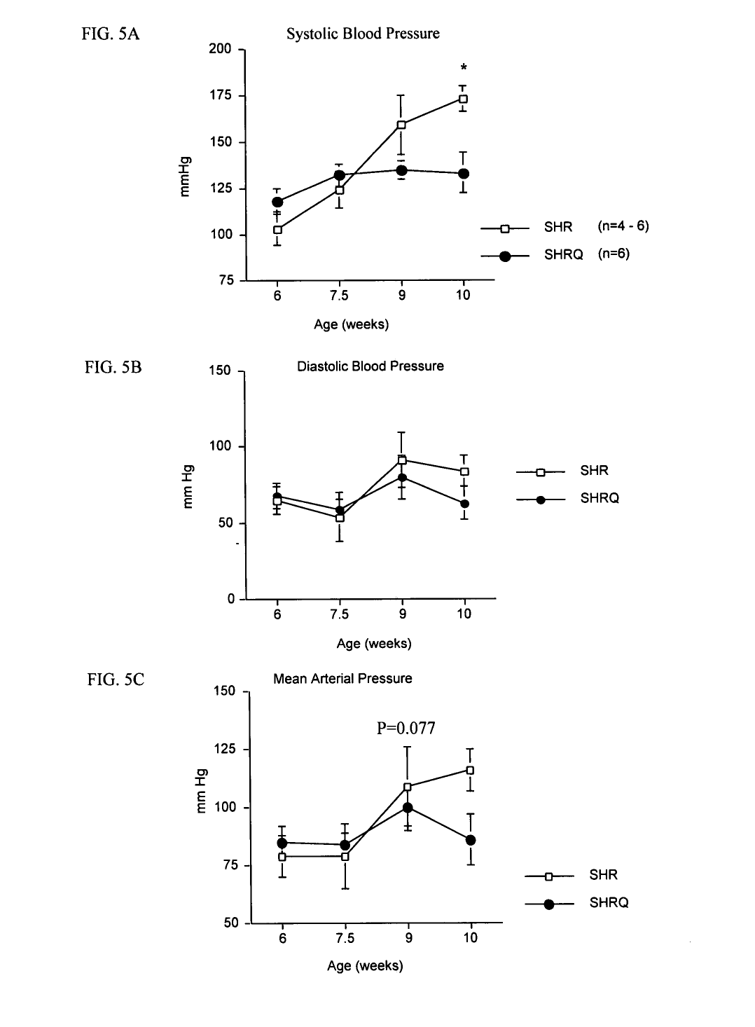

Male SH rats (4 weeks of age) (purchased from Harlan, Indianapolis, Ind.) were measured for baseline blood pressures and body weights and randomly divided into dietary groups; SH rat+control diet (SHR) (n=4 to 6), and SH rat+1.5 g quercetin/kg diet (SHRQ) (n=6) (see, FIGS. 5A and 5C). Blood pressure blood pressure was measured for each group at 6, 7.5, 9 and 10 weeks. Dietary supplement was initiated at 5 weeks and maintained throughout the experiment. This experiment demonstrates that quercetin delayed the onset of hypertension in this genetic model of hypertension.

In a separate experiment using SH rats (dietary supplement was likewise initiated at 5 weeks), blood pressure was measured at 17 weeks. No significant difference in blood pressure was observed between control and quercetin treated rats at 17 weeks. However, it is believed that by 17 weeks the severity of the phenotype in this genetic model system masks the effect of quercetin treatment.

### EXAMPLE VIII

Randomized, placebo-controlled crossover trials were performed to test the hypothesis that quercetin reduces blood pressure in prehypertensive and stage 1 hypertensive human subjects. Systemic markers of oxidant load also were examined as secondary outcomes to determine if reductions in blood pressure were associated with lower indices of oxidative stress.

Participants and Recruitment Criteria.

This study was approved by the University of Utah Human Use Review Committee, University of Utah Institutional Review Board, and written informed consent was obtained from each participant. Recruitment efforts in the greater Salt Lake City area targeted males and females with prehypertension (120-139 mmHg systolic/80-89 mmHg diastolic) and Stage 1 hypertension (140-159 mmHg systolic/90-99 mmHg diastolic) as defined by The 7th Report Of The Joint National Committee On Prevention, Detection, Evaluation, And Treatment Of High Blood Pressure (32).

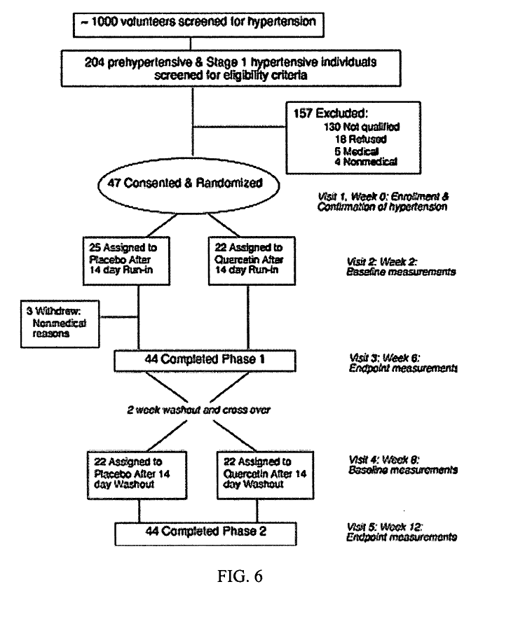

FIG. 6 summarizes the number of subjects screened, recruited, and enrolled in this study. Initial screening consisted of asking volunteers if they had a history of high blood pressure, followed by a single blood pressure measurement using an Omron random zero blood pressure analyzer. If blood pressure criteria were met during the initial screening, subjects were referred to the Nutrition Clinic for further evaluation of blood pressure and confirmation of eligibility criteria. Subjects who met blood pressure guidelines and eligibility criteria after the clinical evaluation were enrolled in the study.

Participants were recruited from October 2004 to June 2005. Forty-seven patients who met study criteria consented and were enrolled, n=24 prehypertensive, and n=23 stage 1 hypertensive. Forty-four subjects completed the entire protocol, 3 withdrew; one male and one female from the prehypertensive group, and one male from the stage 1 hypertensive group. Subjects were not excluded based on their current use of antihypertensive medication, as long as their initial blood pressure was within study limits. Major exclusion criteria for hypertensive subjects included current smoking, history of a prior cardiovascular event, diabetes, renal insufficiency, hyperlipidemia (total cholesterol >240 mg/dl), pregnancy, lactation, any chronic disease that might interfere with study participation, body mass index (BMI) above 35 kg/m², consumption of more than 12 alcoholic drinks weekly, or unwillingness to stop current supplement intake or use of calcium/magnesium antacids. All subjects who met exclusion criteria agreed to maintain their typical diet and exercise habits.

Objectives, Interventions, Outcomes.

The primary hypothesis to be tested was that 365 mg quercetin aglycone/twice per day reduces blood pressure in prehypertensive and stage 1 hypertensive subjects. The secondary hypothesis was that quercetin-induced reductions in blood pressure would be associated with lower indices of systemic oxidant stress.

Participants chosen from the initial screening process were scheduled for a familiarization visit to the clinic at the University of Utah Nutrition Laboratory. During that visit participant responsibilities were explained, consent was obtained, and blood pressure status was verified. Subjects were then instructed to discontinue any existing supplement use and complete a 14-day run-in period (FIG. 6).

After the run-in period, they were enrolled in a double-blind, placebo-controlled, crossover trial consisting of a 4-week quercetin supplementation phase and a 4-week placebo phase. A 14-day washout period separated the two phases. Subjects were randomly assigned to begin either the quercetin or the placebo phase first. Four-week treatment phases were chosen because this duration has been shown to be efficacious concerning dietary interventions (33). Clinic visits were conducted on overnight-fasted subjects in the morning hours at the beginning and end of the placebo and quercetin supplementation phases. Subjects were instructed not to exercise prior to their appointments. Compliance was confirmed by a tablet count at the completion of each phase of the study and by quantifying plasma quercetin concentrations.

Since no human studies have examined whether quercetin reduces blood pressure in hypertensive humans, the dose of quercetin used in this trial was based on efficacious results obtained using animal models of hypertension (5, 31, 34). Tablets containing placebo or quercetin were manufactured by USANA Health Sciences (Salt Lake City, Utah) and quality control analyses of a random sample of quercetin and placebo tablets are summarized in Table 3.

TABLE 3
Composition of quercetin and placebo tablets.

| | Placebo | Quercetin |
|---|---|---|
| | (mg/tablet) | (mg/tablet) |
| Quercetin w/ starch | 0.00 | 364.00 |
| Microcrystalline Cellulose | 564.00 | 192.00 |
| Dicalcium Phosphate | 312.00 | 312.00 |
| Colloidal Silicon Dioxide | 6.00 | 6.00 |
| Ascorbyl Palmitate | 6.00 | 14.00 |
| Croscarmellose Sodium | 12.00 | 12.00 |

Blood Pressure Measurement.

Blood pressure was a primary outcome variable and was obtained at each clinic visit by a trained observer using an Omron random zero automatic blood pressure analyzer as previously described (35). Each participant sat quietly for 5-10 minutes, after which their arm was placed at heart level and blood pressure and pulse rate were determined at least three times in 3-5 minute intervals. If blood pressure varied among these determinations by more than 10 mmHg, three additional trials were performed. The accumulated measurements then were averaged to determine overall systolic and diastolic pressure and pulse rate for each subject.

Venous Blood Collection.

Blood samples were collected after blood pressure was measured. Blood was collected by antecubital venipuncture from fasting subjects into sodium heparin tubes (Becton Dickinson, N.J., USA). Collected blood was immediately stored on ice and centrifuged within 10 minutes at 4° C. for 15 minutes at 2500 g as previously described (36). Plasma was separated and stored at −80° C. until analyzed for quercetin concentration, plasma antioxidant reserve, and ferric reducing antioxidant power.

Blood Lipid, Glucose, and Urine Collection.

During each patient visit whole blood (˜50 μL) was obtained from a digit puncture to determine blood lipids (LDL, VLDL, HDL, total cholesterol) and glucose using a clinical Cholestech LDX blood analyzer (37). Prior to each patient visit, first morning urine was collected, brought to the laboratory, and stored at −80° C. for later analysis of isoprostane concentrations.

Quercetin Analysis.

Plasma quercetin was analyzed as previously described (38) with slight modifications; 250 μL samples were hydrolyzed with 100 μL and 500 μL of 6 N HCl. Supernatants then were extracted with ethyl acetate and injected into an HPLC Discovery C18 column. The mobile phase was 1% acetic acid (solvent A) and 95% acetonitrile in 1% phosphoric acid (solvent B). The gradient elution was solvent B from 10 to 85% in 20 minutes, holding for 5 minutes before returning back to 10% for conditioning. A visible detector with 365 nm was used. Quercetin was quantified by peak height ratio method and expressed as ng/mL serum.

Indices of Oxidative Stress.

Plasma antioxidant reserve (PAR).

Ex-vivo amplification of isoprostanes was accomplished by introducing a source of free radicals (3-morpholinosydnonimine, SIN-1) into the blood plasma to induce oxidation of lipoproteins (39). Uric acid, a major water soluble antioxidant present in plasma, was removed using uricase prior to introduction of SIN-1 so that any protection provided by other antioxidants could be measured. Therefore, PAR measures the non-urate antioxidant capacity, or antioxidant power, of blood. It has been previously demonstrated that supplementing antioxidants can increase the antioxidant capacity of the blood as determined by PAR (39).

Ferric Reducing Antioxidant Power (FRAP).

FRAP measures the ability of an antioxidant to reduce Fe³⁺ to Fe²⁺ (40), and is an index of plasma antioxidant potential in hypertensive patients (41-44). This assay was done as previously described (39). Briefly, plasma samples were diluted 1:2 with phosphate buffered saline, followed by addition of a reagent solution containing 0.8 mmol/L 2,4,6-tri-(2-pyridyl)-s-tirazine and 1.7 mmol/l FeCl₃.6-H2O. Samples were then incubated at 37° C. for 15 min and the absorbance at 593 nm was recorded in a plate reader (Molecular Devices, Spectramax 340 pc).

Urinary Isoprostane Measurement.

Urine 8-isoprostane F2α is a measure of lipid peroxidation and can be used to estimate oxidative stress in humans (45-47). Quantification of 8-isoprostane F2α (also known as 8-epi-PGF2α or 8-iso-PGF2α) in urine samples was performed using a competitive enzyme-linked immunoassay kit (Cayman Chemical, Ann Arbor, Mich.) according to the manufacturers instructions.

Statistical Analyses.

All data are reported as means±SEM. All variables were analyzed using paired t tests to detect differences before and after the placebo phase, and before and after the quercetin phase (SPSS v.11.0.3). Dietary intake data and plasma quercetin concentrations were examined using paired t tests comparing placebo vs. quercetin phases. Significance was accepted at P<0.05.

Results

Patient Characteristics.

Nearly 1000 patients were interviewed and screened for eligibility. The majority (i.e. <700) were not considered further for participation because they met one of more of the exclusion criteria. From this initial screening, 204 were evaluated in more detail at a subsequent clinic visit to determine if all inclusion/exclusion criteria were met, and that blood pressure was within the study limits (FIG. 6). Forty-seven subjects were initially enrolled and 44 completed the entire 12-week study. Subject characteristics are summarized in Table 4. No adverse effects of quercetin or placebo treatment were reported during the course of the study. Weight and BMI did not change between treatments in either group (Table 5). Heart rate was unchanged throughout the study (data not shown).

TABLE 4
Subject characteristics.

| | | Stage 1 |
|---|---|---|
| | Prehypertensive | Hypertensive |
| n | 22 | 22 |
| Age, y | 47.8 ± 3.5 | 49.2 ± 2.9 |
| Male sex | n = 13 | n = 13 |
| Caucasian | n = 18 | n = 22 |
| Asian | n = 2 | n = 0 |
| Hispanic | n = 1 | n = 0 |
| Polynesian | n = 1 | n = 0 |
| Diuretic use (“DU”) | n = 2 | n = 0 |
| Angiotensin II receptor agonist use | n = 1 | n = 0 |
| ACE + DU + Ca channel blocker | n = 1 | n = 0 |
| ACE inhibitor + DU | n = 1 | n = 0 |

TABLE 5
Body mass and parameters of oxidative stress¹.

| | | | | |
|---|---|---|---|---|
| | | Urinary | | |
| | | Isoprostane | | |
| Weight | BMI | (ng/g | FRAP² | PAR³ |
| (kg) | (kg/m²) | creatine) | (μmol/L) | (pg/mL) |

Prehypertensive - Placebo

| | | | | | |
|---|---|---|---|---|---|
| Baseline | 88.4 ± 5.0 | 29.8 ± 1.4 | 541 ± 75 | 1161 ± 59 | 322 ± 32 |
| Endpoint | 88.3 ± 5.0 | 29.7 ± 1.4 | 588 ± 92 | 1055 ± 68 | 299 ± 51 |
| P value | 0.80 | 0.93 | 0.57 | 0.20 | 0.50 |
| n | 22 | 22 | 10 | 10 | 10 |

Prehypertensive - Quercetin

| | | | | | |
|---|---|---|---|---|---|
| Baseline | 88.4 ± 5.0 | 29.6 ± 1.4 | 518 ± 55 | 1080 ± 48 | 279 ± 48 |
| Endpoint | 88.4 ± 4.9 | 29.8 ± 1.4 | 520 ± 75 | 1106 ± 61 | 266 ± 32 |
| P value | 0.82 | 0.97 | 0.98 | 0.55 | 0.71 |
| n | 22 | 22 | 10 | 10 | 10 |

Stage 1 Hypertensive - Placebo

| | | | | | |
|---|---|---|---|---|---|
| Baseline | 88.7 ± 4.4 | 29.5 ± 1.4 | 592 ± 105 | 1096 ± 70 | 285 ± 60 |
| Endpoint | 88.0 ± 4.4 | 29.4 ± 1.4 | 911 ± 355 | 1086 ± 66 | 238 ± 52 |
| P value | 0.07 | 0.40 | 0.251 | 0.751 | 0.177 |
| n | 22 | 22 | 8 | 8 | 8 |

Stage 1 Hypertensive - Quercetin

| | | | | | |
|---|---|---|---|---|---|
| Baseline | 88.7 ± 5.5 | 29.3 ± 1.3 | 822 ± 249 | 1056 ± 59 | 258 ± 55 |
| Endpoint | 88.2 ± 5.5 | 29.5 ± 1.4 | 549 ± 59 | 1152 ± 69 | 281 ± 74 |
| P value | 0.10 | 0.24 | .265 | 0.107 | 0.487 |
| n | 22 | 22 | 8 | 8 | 8 |

Values are reported as mean ± SEM, paired T-tests used to compared baseline vs. endpoint for both placebo and quercetin treatment in each experimental group.
²FRAP, Ferric reducing antioxidant potential of plasma.
³Plasma antioxidant reserve.

Plasma quercetin (ng/ml) was 235±35 after placebo-treatment and increased to 480±64 after quercetin-treatment. To validate that a 2-week washout period was sufficient to reduce plasma quercetin to baseline, 5 subjects consumed quercetin supplements for 1 week, followed by a 1-week washout period. After this washout period, plasma quercetin concentrations were 190±9 ng/mL. These values are similar to those obtained from subjects that consumed placebo and had not yet been exposed to quercetin.

In order to rule out the possibility that the antihypertensive effect of quercetin persisted even after plasma levels return to baseline, mean arterial pressure at baseline of the quercetin phase and placebo phase was evaluated in those subjects that received quercetin treatment first. No differences were found, demonstrating that carryover antihypertensive effects were not present in subjects receiving quercetin treatment before placebo.

Blood Pressure.

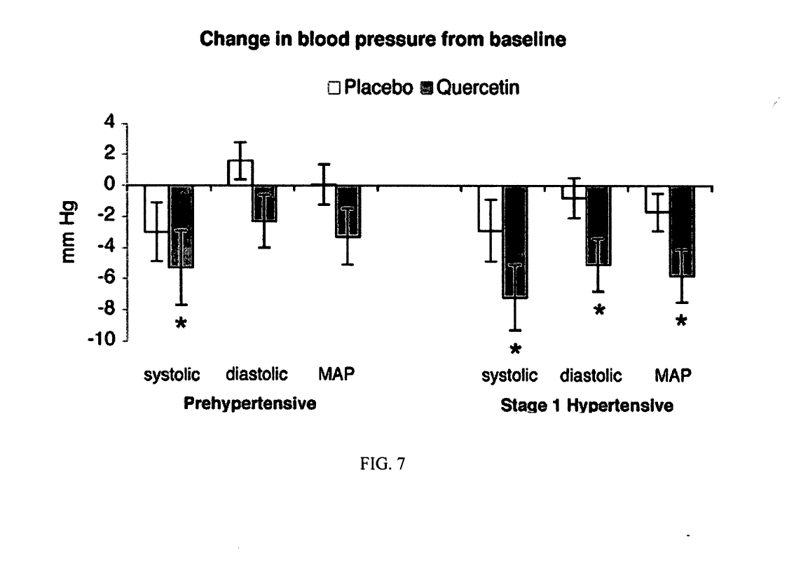

Placebo treatment did not alter blood pressure in either group of hypertensive subjects. In contrast, quercetin supplementation reduced systolic pressure in prehypertensives, while systolic, diastolic, and mean arterial pressure were decreased in stage 1 hypertensive subjects (Table 6, FIG. 7). Regression analyses demonstrated the antihypertensive effect of quercetin to be independent of age and gender in both groups of hypertensive subjects. During the study, subjects were randomly assigned to begin either the placebo or quercetin phase first. There was no effect of this treatment order on the observed changes in blood pressure.

TABLE 6
Blood Pressure¹.

| | Systolic | Diastolic | MAP² |
|---|---|---|---|
| | (mmHg) | (mmHg) | (mmHg) |
| PREHYPERTENSIVE | | | |
| Blood pressure at enrollment | 137 ± 2 | 86 ± 1 | 103 ± 1 |
| Placebo | | | |
| Baseline | 135 ± 3 | 84 ± 1 | 101 ± 1 |
| Endpoint | 132 ± 2 | 86 ± 1 | 101 ± 1 |
| P value | 0.12 | 0.19 | 0.96 |
| Quercetin | | | |
| Baseline | 133 ± 2 | 85 ± 1 | 101 ± 1 |
| Endpoint | 128 ± 3* | 83 ± 2 | 98 ± 2 |
| P value | 0.04 | 0.19 | 0.09 |
| STAGE 1 HYPERTENSIVE | | | |
| Blood pressure at enrollment | 148 ± 2 | 96 ± 1 | 113 ± 1 |
| Placebo | | | |
| Baseline | 141 ± 2 | 94 ± 2 | 110 ± 2 |
| Endpoint | 138 ± 2 | 93 ± 2 | 108 ± 2 |
| P value | 0.17 | 0.41 | 0.17 |
| Quercetin | | | |
| Baseline | 145 ± 2 | 97 ± 1 | 113 ± 1 |
| Endpoint | 138 ± 2* | 92 ± 2* | 108 ± 2* |
| P value | <0.01 | <0.01 | <0.01 |

¹Values are reported as mean ± SEM, paired T-tests used to compared baseline vs. endpoint for both placebo and quercetin treatment in each experimental group.
²MAP, mean arterial pressure.

Indices of Oxidative Stress and Antioxidant Capacity.

Measurements of antioxidant capacity (fasting plasma FRAP, PAR) and oxidative stress (fasting urinary 8-isoprostane F2α concentration) were not altered by quercetin supplementation vs. placebo (Table 5). Regression analyses indicated that the antihypertensive effect of quercetin was independent of PAR, FRAP, and urinary 8-isoprostane F2α.

Dietary Analyses.

Three-day diet record analysis indicated that there was lower potassium and magnesium intake during the quercetin phase vs. placebo in prehypertensive patients (Table 7). Stage 1 hypertensive subjects had reductions in vitamin A and potassium intake during the quercetin supplementation vs. placebo phase (Table 7). All other nutrients evaluated were similar in placebo vs. quercetin phases in both groups of hypertensive subjects.

TABLE 7
Analysis of 3-day dietary records¹.

| | Prehypertensive (n = 20) | | | Stage 1 hypertensive (n = 21) | | |
|---|---|---|---|---|---|---|
| | Placebo | Quercetin | P | Placebo | Quercetin | P |
| Kilocalorie | 2126 ± 123 | 2236 ± 185 | 0.56 | 2096 ± 154 | 1948 ± 148 | 0.36 |
| Protein, g | 89 ± 5 | 88 ± 7 | 0.88 | 87 ± 5 | 83 ± 6 | 0.42 |
| Fat, g | 74 ± 6 | 81 ± 8 | 0.41 | 80 ± 6 | 72 ± 6 | 0.36 |
| Saturated Fat, g | 25 ± 2 | 26 ± 3 | 0.62 | 24 ± 2 | 22 ± 2 | 0.27 |
| Polyunsaturated Fat, g | 9 ± 1 | 9 ± 1 | 0.92 | 12 ± 2 | 10 ± 2 | 0.86 |
| Monounsaturated Fat, g | 19 ± 2 | 18 ± 2 | 0.49 | 19 ± 2 | 18 ± 2 | 0.21 |
| Fiber, g | 21 ± 1 | 22 ± 2 | 0.32 | 22 ± 3 | 22 ± 4 | 1.00 |
| Carbohydrate, g | 282 ± 18 | 295 ± 26 | 0.61 | 252 ± 27 | 244 ± 23 | 0.79 |
| Cholesterol, mg | 284 ± 34 | 287 ± 35 | 0.86 | 232 ± 28 | 221 ± 28 | 0.60 |
| Vitamin A, IU | 7796 ± 984 | 8637 ± 1822 | 0.64 | 7615 ± 1103 | 5560 ± 799* | 0.03 |
| Vitamin C, mg | 165 ± 26 | 138 ± 17 | 0.28 | 164 ± 56 | 103 ± 16 | 0.29 |
| Vitamin E, IU | 13 ± 2 | 16 ± 3 | 0.28 | 15 ± 4 | 14 ± 3 | 0.79 |
| Vitamin K, μg | 62 ± 14 | 110 ± 29 | 0.06 | 64 ± 22 | 43 ± 8 | 0.28 |
| Calcium, mg | 843 ± 78 | 816 ± 77 | 0.58 | 1039 ± 95 | 966 ± 128 | 0.50 |
| Magnesium, mg | 283 ± 39 | 250 ± 41* | 0.03 | 276 ± 24 | 261 ± 29 | 0.52 |
| Sodium, mg | 2973 ± 282 | 3383 ± 422 | 0.36 | 2794 ± 253 | 3153 ± 238 | 0.14 |
| Potassium, mg | 2919 ± 189 | 2490 ± 191* | 0.05 | 2465 ± 239 | 2195 ± 234* | 0.05 |
| Selenium, μg | 77 ± 7 | 65 ± 8 | 0.20 | 86 ± 12 | 79 ± 12 | 0.34 |
| Zinc, mg | 10 ± 1 | 9 ± 1 | 0.17 | 9 ± 1 | 8 ± 1 | 0.36 |

Values are mean ± SEM, paired T-test used to compare Placebo vs. Quercetin.

Fasting Plasma Lipids, and Glucose.

These outcomes were quantified because earlier studies have reported beneficial changes in the blood lipid profile of quercetin supplemented rats that consumed a cholesterol rich diet (49). There was no change in total cholesterol, triglycerides, LDL, HDL, or total cholesterol: HDL cholesterol ratio after quercetin supplementation compared to placebo in prehypertensive or stage 1 hypertensive patients (data not shown). Fasting blood glucose levels also were also unchanged (data not shown).

Discussion

Results from this investigation support the primary hypothesis and are the first to demonstrate that daily supplementation with 730 mg quercetin for 28 days reduces systolic blood pressure in prehypertensive individuals, and both systolic and diastolic pressure in subjects with stage 1 hypertension. These findings are an important extension of previous studies showing that quercetin lowers blood pressure in hypertensive animals (5, 30, 31, 50, 51), and prevents the onset of hypertension in response to mechanical overload in rodents (30). The antihypertensive effect of quercetin in the subjects may also explain, at least in part, why previous epidemiological reports show an inverse relationship between dietary flavonoid intake and heart disease risk (21-28, 52-56). In contrast to results obtained from in vitro experiments and animal models (5, 30, 31, 50, 51, 57), quercetin-evoked reduction in oxidative stress, as determined by plasma PAR, FRAP and urinary isoprostanes, was not observed.

Only one other study has examined the effect of quercetin supplementation in humans (58). In that investigation, Conquer et al. reported no changes in blood pressure when normotensive individuals (i.e.,<120 systolic/<80 diastolic) were supplemented with 1000 mg/day of quercetin for one month, in spite of similar plasma quercetin concentrations (427±89 ng/ml) compared to the present study (480±64 ng/ml) (58). These data indicate a certain degree of hypertension might be required in order for quercetin to exert a blood pressure lowering effect. This possibility is supported by data from the present study wherein quercetin reduced systolic, diastolic and mean arterial pressure blood pressure in stage 1 hypertensive subjects, but only systolic blood pressure in those with prehypertension. Likewise, animal based studies have demonstrated that quercetin is efficacious in lowering blood pressure in hypertensive, but not normotensive rats (5, 30).

Three-day diet records were used to evaluate whether changes in nutrient intake influenced blood pressure during the quercetin treatment phase. Though stage 1 hypertensive subjects had slightly decreased intakes of vitamin A and potassium, and prehypertensives consumed less potassium and magnesium during the quercetin phase, it is unlikely that these changes led to reduced blood pressure. With regard to dietary intake of polyphenolic compounds, it is not possible to determine intake of items such as quercetin since there are no suitable databases available for such an analysis. The lack of databases likely can be attributed to the variation in chemical composition of fruits and vegetables, coupled with the lack of sufficiently accurate analytical tools (52). In spite of these limitations, it has been estimated that average dietary intake of quercetin from Western diets is 28-42 mg/day (52, 54, 55). It is likely that the intake of polyphenolic compounds in individual diets is dependent on fruit, vegetable, and whole grain consumption, and variations in these foods would be reflected in the vitamin, mineral and fiber content reported. Since these dietary variables were generally similar among subjects during the placebo and quercetin-phases of the study, it is believed that intake of polyphenolic compounds was not different among participants in the investigation.

Recent American Heart Association statistics estimate that over 50 million Americans suffer from hypertension (57). There is a linear relationship between blood pressure and mortality from stroke and ischemic heart disease that emphasizes the importance of blood pressure control (59). Based on risk assessment summarized in the 7th Report of the Joint National Committee on Prevention, Detection, Evaluation, and Treatment of High Blood Pressure, the risk of death from ischemic heart disease and stroke in prehypertensive patients used in the present study is double that of individuals with blood pressure of 115/75 mmHg, and nearly four-times greater in stage 1 hypertensive patients (32). The quercetin induced-lowering of systolic blood pressure observed in prehypertensive (−5.2 mmHg) and stage 1 hypertensive (−7.2 mmHg) are clinically relevant because reductions of this magnitude are associated with a decrease in mortality of ˜14% and ˜9% from stroke and coronary heart disease, respectively (59). These findings are noteworthy in light of the emergence of systolic blood pressure as a more important risk factor than diastolic pressure with regard to mortality from cardiovascular disease, particularly in individuals over 50 years of age (32).

Lifestyle modification has been emphasized in pre-hypertensive individuals as an initial intervention to control blood pressure (32). Interestingly, the reduction of blood pressure observed in stage 1 hypertensive subjects after quercetin supplementation is similar to those experienced following sodium reduction, weight reduction, increased physical activity, or alcohol reduction (60). Other proven lifestyle modifications such as the DASH (Dietary Approaches to Stop Hypertension) diet result in similar or slightly greater blood pressure reduction (60). Thus, it appears that the effects of quercetin supplementation are consistent with current recommended lifestyle modifications used to reduce blood pressure.

The secondary hypothesis of the human experiments was that the antihypertensive effect of quercetin would be associated with a reduction of systemic oxidant stress. Rationale for this hypothesis was based on studies showing that quercetin lowers indices of oxidative stress in a dose-dependent manner in spontaneously hypertensive rats (e.g., lower urinary isoprostanes, plasma malondialdehyde) (5) and nitric-oxide deficient rats (e.g., reduced plasma malondialdehyde and glutathione peroxidase activity) (31). Instead, it was observed that plasma antioxidant potential (FRAP and PAR analyses) and urinary 8-isoprostane F2α were similar among prehypertensive and stage 1 hypertensive subjects regardless of quercetin or placebo treatment. It is believed that the lack of an antioxidant effect with quercetin treatment is not attributed to the dose used. The dose was similar (730 mg/day, ˜8.5 mg/kg) to the dose used in previous animal studies which did show antioxidant effects (10 mg/kg) (5, 31, 34). Nevertheless, species-dependent differences in metabolism of quercetin (human vs. rat) may exist. An important consideration, however, is the severity of oxidant stress in the hypertensive subjects. In this regard, plasma FRAP (μmol/L) was similar between both groups of hypertensive subjects (1065-1130) from the present trial and normotensive subjects (973-1064) that were evaluated in a previous study (39). These data indicate that the cohort evaluated in the present investigation did not have elevated oxidant stress, at least in terms of FRAP. As such, the ability of quercetin to further reduce markers of oxidative stress measured may be limited.

Evidence does exist, however, for a mechanism involving angiotensin converting enzyme (ACE). For example, 30 mg/kg quercetin (p.o.) in rats significantly blunted the hypertensive response to i.v. administration of angiotensin II (61). While it is possible that higher systemic concentrations of quercetin, as observed in the present study, could limit angiotensin II production and lower blood pressure, further investigation would be required to confirm this speculation.

The present study is the first to show that quercetin reduces blood pressure in prehypertensive and Stage I hypertensive individuals. A powerful experimental design (double-blinded, placebo-controlled, crossover) was used and found quercetin supplementation to be efficacious in reducing blood pressure. The data indicate that potential exists for this polyphenolic compound to be used as adjunct therapy in diet/lifestyle interventions to help control blood pressure in prehypertensive and stage 1 hypertensive individuals.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

All references, including publications, patents, and patent applications, cited herein are hereby incorporated by reference to the same extent as if each reference were individually and specifically indicated to be incorporated by reference and were set forth in its entirety herein. The following list of references is hereby incorporated by reference in their entirety:

1. Melina D, Coliricci F, Buerra G, Melina G, Frustaci A, Caldarulo M, Guerrera C. Prevalence of left ventricular hypertrophy and cardiac arrythmias in borderline hypertension. American Journal of Hypertension 5(8):570-573, 1992;

2. Brody S, Preut R, Schommer K, Schurmeyer T H. A randomized controlled trial of high dose ascorbic acid for reduction of blood pressure, cortisol, and subjective responses to psychological stress. Psychopharmacology. 2002;159:319-24;

3. Aviram M, Dornfeld L. Pomegranate juice consumption inhibits serum angiotensin converting enzyme activity and reduces systolic blood pressure. Atherosclerosis. 2001;158:195-8;

4. Koba K, Abe K, Ikeda I, Sugano M. Effects of alpha-tocopherol and tocotrienols on blood pressure and linoleic acid metabolism in the spontaneously hypertensive rat (SHR). Biosci Biotechnol Biochem. 1992;56:1420-3;

5. Duarte J, Perez-Palencia R, Vargas F, Ocete M A, Perez-Vizcaino F, Zarzuelo A, Tamargo J. Antihypertensive effects of the flavonoid quercetin in spontaneously hypertensive rats. Br J Pharmacol. 2001;133:117-24;

6. Graefe E U, Wittig J, Mueller S, Riethling A K, Uehleke B, Drewelow B, Pforte H, Jacobasch G, Derendorf H, Veit M. Pharmacokinetics and bioavailability of quercetin glycosides in humans. J Clin Pharmacol. 2001;41:492-9;

7. Makita N, Yasuda H. Alterations of phosphoinositide-specific phospholipase C and protein kinase C in the myocardium of spontaneously hypertensive rats. Basic Res Cardiol. 1990;85:435-43;

8. Rizzoni D, Castellano M, Porteri E, Bettoni G, Muiesan M L, Agabiti-Rosei E. Vascular structural and functional alterations before and after the development of hypertension in SHR. Am J Hypertens. 1994;7:193-200;

9. Roba J L. The use of spontaneously hypertensive rats for the study of anti-hypertensive agents. Lab Anim Sci. 1976;26:305-19;

10. Pedro-Botet J, Covas M I, Martin S, Rubies-Prat J. Decreased endogenous antioxidant enzymatic status in essential hypertension. J Hum Hypertens. 2000;14:343-5.

11. Vaziri N D, Wang X Q, Oveisi F, Rad B. Induction of oxidative stress by glutathione depletion causes severe hypertension in normal rats. Hypertension. 2000;36: 142-6;

12. Vaziri N D, Liang K, Ding Y. Increased nitric oxide inactivation by reactive oxygen species in lead-induced hypertension. Kidney Int. 1999;56:1492-8;

13. Newaz M A, Nawal N N. Effect of gamma-tocotrienol on blood pressure, lipid peroxidation and total antioxidant status in spontaneously hypertensive rats (SHR). Clin Exp Hypertens. 1999;21:1297-313;

14. Galley H F, Thornton J, Howdle P D, Walker B E, Webster N R. Combination oral antioxidant supplementation reduces blood pressure. Clin Sci (Colch). 1997;92:361-5;

15. Vaziri N D, Ni Z, Oveisi F, Trnavsky-Hobbs D L. Effect of antioxidant therapy on blood pressure and NO synthase expression in hypertensive rats. Hypertension. 2000;36:957-64;

16. Naguib Y M. A fluorometric method for measurement of oxygen radical-scavenging activity of water-soluble antioxidants. Anal Biochem. 2000;284:93-8;

17. Jalili T, Takeishi Y, Song G, Ball N A, Howles G, Walsh R A. PKC translocation without changes in Galphaq and PLC-beta protein abundance in cardiac hypertrophy and failure. Am J Physiol. 1999;277:H2298-304;

18. Jalili, T., Y. Takeishi, G. Song, N. A. Ball, G. Howles, and R. A. Walsh. 1999. PKC translocation without changes in Galphaq and PLC-beta protein abundance in cardiac hypertrophy and failure, Am J Physiol 277:H2298-2304;

19. Takeishi, Y., Q. Huang, J. Abe, M. Glassman, W. Che, J. D. Lee, H. Kawakatsu, E. G. Lawrence, B. D. Hoit, B. C. Berk, and R. A. Walsh. (2001) Src and multiple MAP kinase activation in cardiac hypertrophy and congestive heart failure under chronic pressure-overload: comparison with acute mechanical stretch, J Mol Cell Cardiol 33:1637-48; and

20. OKAMOTO, K. and AOKI, K. (1963). Development of a strain of spontaneously hypertensive rats, Jap. Circ. J. 27:282-293.

21. Hertog, M. G. and Hollman, P. C. (1996). Potential health effects of the dietary flavonol quercetin, Eur. J. Clin. Nutr. 50:63-71.

22. Knekt, P., Kumpulainen, J., Jarvinen, R., et al. (2002). Flavonoid intake and risk of chronic diseases, Am J Clin Nutr 76:560-8.

23. Knekt, P., Jarvinen, R., Reunanen, A., Maatela, J. (1996). Flavonoid intake and coronary mortality in Finland: a cohort study Bmj 312:478-81.

24. Constant, J. (1997). Alcohol, ischemic heart disease, and the French paradox, Coron Artery Dis 8:645-9.

25. Keli, S. O., Hertog, M. G., Feskens, E. J., Kromhout, D. (1996). Dietary flavonoids, antioxidant vitamins, and incidence of stroke: the Zutphen study, Arch Intern Med 156:637-42.

26. Hertog, M. G., Feskens, E. J., Hollman, P. C., Katan, M. B., Kromhout, D. (1993). Dietary antioxidant flavonoids and risk of coronary heart disease: the Zutphen Elderly Study, Lancet 342:1007-11.

27. Huxley, R. R. and Neil, H. A. (2003). The relation between dietary flavonol intake and coronary heart disease mortality: a meta-analysis of prospective cohort studies, Eur J Clin Nutr 57:904-8.

28. Mennen, L. I., Sapinho, D., de Bree, A., et al. (2004). Consumption of foods rich in flavonoids is related to a decreased cardiovascular risk in apparently healthy French women, J Nutr 134:923-6.

29. Duarte, J., Perez-Vizcaino, F., Zarzuelo, A., Jimenez, J., Tamargo, J. (1993). Vasodilator effects of quercetin in isolated rat vascular smooth muscle, Eur J Pharmacol 239:1-7.

30. Jalili, T., Carlstrom, J., Kim, S., et al. (2006). Quercetin-supplemented diets lower blood pressure and attenuate cardiac hypertrophy in rats with aortic constriction, J Cardiovasc Pharmacol 47:531-41.

31. Duarte, J., Jimenez, R., O'Valle, F., et al. (2002) Protective effects of the flavonoid quercetin in chronic nitric oxide deficient rats, J Hypertens 20:1843-54.

32. Chobanian, A. V., Bakris, G. L., Black, H. R., et al. (2003). Seventh report of the Joint National Committee on Prevention, Detection, Evaluation, and Treatment of High Blood Pressure, 42:1206-52.

33. Appel, L. J., Moore, T. J., Obarzanek, E., et al. (1997). A clinical trial of the effects of dietary patterns on blood pressure, DASH Collaborative Research Group, N Engl J Med, 336:1117-24.

34. Carlstrom, J., Symons, J. D., Wu, T. C., Bruno, R. S., Litwin, S. E., Jalili, T. (2007). A quercetin supplemented diet does not prevent cardiovascular complications in spontaneously hypertensive rats, J Nutr 137:628-33.

35. Redon J, Oliva M R, Tormos C, et al. Antioxidant activities and oxidative stress byproducts in human hypertension. Hypertension 2003;41:1096-101.

36. Vassalle C, Botto N, Andreassi M G, Berti S, Biagini A. Evidence for enhanced 8-isoprostane plasma levels, as index of oxidative stress in vivo, in patients with coronary artery disease. Coron Artery Dis 2003;14:213-8.

37. Bard R L, Kaminsky L A, Whaley M H, Zajakowski S. Evaluation of lipid profile measurements obtained from the Cholestech L.D.X analyzer. J Cardiopulm Rehabil 1997;17:413-8.

38. Maiani G, Serafini M, Salucci M, Azzini E, Ferro-Luzzi A. Application of a new high-performance liquid chromatographic method for measuring selected polyphenols in human plasma. J Chromatogr B Biomed Sci Appl 1997;692:311-7.

39. Rabovsky A, Cuomo J, Eich N. Measurement of plasma antioxidant reserve after supplementation with various antioxidants in healthy subjects. Clin Chim Acta 2006;371:55-60.

40. Benzie I F, Strain J J. The ferric reducing ability of plasma (FRAP) as a measure of “antioxidant power”: the FRAP assay. Anal Biochem 1996;239:70-6.

41. Kashyap M K, Yadav V, Sherawat B S, et al. Different antioxidants status, total antioxidant power and free radicals in essential hypertension. Mol Cell Biochem 2005;277:89-99.

42. Lopes H F, Martin K L, Nashar K, Morrow J D, Goodfriend T L, Egan B M. DASH diet lowers blood pressure and lipid-induced oxidative stress in obesity. Hypertension 2003;41 :422-30.

43. Skalska A, Gasowski J, Stepniewski M, Grodzicki T. Antioxidative protection in hypertensive patients treated with diuretics. Am J Hypertens 2005; 18:1130-2.

44. Vassalle C, Masini S, Carpeggiani C, L'Abbate A, Boni C, Zucchelli G C. In vivo total antioxidant capacity: comparison of two different analytical methods. Clin Chem Lab Med 2004;42:84-9.

45. Cracowski J L, Baguet J P, Ormezzano O, et al. Lipid peroxidation is not increased in patients with untreated mild-to-moderate hypertension. Hypertension 2003;41 :286-8.

46. Ward N C, Hodgson J M, Croft K D, Burke V, Beilin L J, Puddey I B. The combination of vitamin C and grape-seed polyphenols increases blood pressure: a randomized, double-blind, placebo-controlled trial. J Hypertens 2005;23:427-34.

47. Ward N C, Hodgson J M, Puddey I B, Mori T A, Beilin L J, Croft K D. Oxidative stress in human hypertension: association with antihypertensive treatment, gender, nutrition, and lifestyle. Free Radic Biol Med 2004;36:226-32.

48. McCullough M L, Karanja N M, Lin P H, et al. Comparison of nutrient databases with chemical composition data from the Dietary Approaches to Stop Hypertension trial. DASH Collaborative Research Group. J Am Diet Assoc 1999;99:S45-53.

49. Igarashi K, Ohmuma M. Effects of isorhamnetin, rhamnetin, and quercetin on the concentrations of cholesterol and lipoperoxide in the serum and liver and on the blood and liver antioxidative enzyme activities of rats. Biosci Biotechnol Biochem 1995;59:595-601.

50. Garcia-Saura M F, Galisteo M, Villar I C, et al. Effects of chronic quercetin treatment in experimental renovascular hypertension. Mol Cell Biochem 2005;270:147-55.

51. Payne J A, Reckelhoff J F, Khalil R A. Role of oxidative stress in age-related reduction of NO-cGMP-mediated vascular relaxation in SHR. Am J Physiol Regul Integr Comp Physiol 2003;285:R542-51.

52. Scalbert A, Williamson G. Dietary intake and bioavailability of polyphenols. J Nutr 2000;130:2073S-85S.

53. Manach C, Williamson G, Morand C, Scalbert A, Remesy C. Bioavailability and bioefficacy of polyphenols in humans. I. Review of 97 bioavailability studies. Am J Clin Nutr 2005;81:230S-242S.

54. de Vries J H, Janssen P L, Hollman P C, van Staveren W A, Katan M B. Consumption of quercetin and kaempferol in free-living subjects eating a variety of diets. Cancer Lett 1997;114:141-4.

55. Formica J V, Regelson W. Review of the biology of Quercetin 1 and related bioflavonoids. Food Chem Toxicol 1995;33:1061-80.

56. Mennen L I, Witteman J C, Geleijnse J M, Stolk R P, Visser M C, Grobbee D E. [Risk factors for cardiovascular diseases in the elderly; the ERGO study (Erasmus Rotterdam Health and the Elderly)]. Ned Tijdschr Geneeskd 1995;139:1983-8.

57. Heart Disease and Stroke Statistics—2004 Update. Dallas, Tex.: American Heart Association, 2003.

58. Conquer J A, Maiani G, Azzini E, Raguzzini A, Holub B J. Supplementation with quercetin markedly increases plasma quercetin concentration without effect on selected risk factors for heart disease in healthy subjects. J Nutr 1998;128:593-7.

59. Lewington S, Clarke R, Qizilbash N, Peto R, Collins R. Age-specific relevance of usual blood pressure to vascular mortality: a meta-analysis of individual data for one million adults in 61 prospective studies. Lancet 2002;360:1903-13.

60. Whelton P K, He J, Appel L J, et al. Primary prevention of hypertension: clinical and public health advisory from The National High Blood Pressure Education Program. Jama 2002;288:1882-8.

61. Hackl L P, Cuttle G, Dovichi S S, Lima-Landman M T, Nicolau M. Inhibition of angiotesin-converting enzyme by quercetin alters the vascular response to brandykinin and angiotensin I. Pharmacology 2002;65:182-6.

What is claimed is:

1. A method for preventing or delaying the onset of or slowing the progression of hypertension in a subject, said method comprising:
administering to a subject a nutritional supplement including quercetin, wherein said supplement is administered in an amount effective in preventing, delaying or slowing the onset or progression of hypertension.

2. The method according to claim 1, wherein an amount effective in preventing, delaying or slowing the onset or progression of hypertension comprises a daily dosage of quercetin of about 15 mg to about 250 mg/kg of body weight.

3. The method according to claim 1, wherein said quercetin is present in an amount to provide a daily dosage of about 0.1 g to about 17.5 g.

4. The method according to claim 1, wherein said supplement additionally contains other nutrients selected from the group consisting of vitamin B₁₂, vitamin B₆, non-flavonoid antioxidants, minerals and trace metals.

5. The method according to claim 1, wherein said supplement additionally contains a non-flavonoid antioxidants selected from the group consisting of selenium, vitamin E, vitamin C, niacin, beta-carotene and coenzyme Q10, or combinations thereof.

6. The method according to claim 5, wherein said supplement additionally contains a non-flavonoid mineral or trace metal selected from the group consisting of zinc, copper, magnesium, manganese, chromium, molybdenum, iron and calcium.

7. The method according to claim 1, wherein said supplement further includes one or more agents selected from the group consisting of a taste-improving agent, coloring agent, preservative, stabilizer, regulator and emulsifier.

8. The method according to claim 1, wherein said nutritional supplement is administered orally as a liquid.

9. The method according to claim 1, wherein said supplement is administered orally as a nutritional food.

10. The method according to claim 1, wherein said hypertension is an agent in the development of left ventricular hypertrophy.

11. The method according to claim 8, wherein said supplement additionally contains vitamin C or vitamin E.

12. The method according to claim 9, wherein said supplement additionally contains vitamin C or vitamin E.

13. The method according to claim 9, wherein said supplement is an energy bar or a cookie.

14. The method according to claim 8, wherein said supplement is a beverage.

15. The method according to claim 1, wherein said supplement is administered in an amount effective in delaying the onset of hypertension.

16. The method according to claim 1, said method further comprising determining if a subject is suffering from hypertension or is prone to the development of hypertension.

17. The method according to claim 14, wherein said beverage is orange juice.

18. A nutritional supplement useful in aiding in preventing, delaying the onset of and/or slowing the progression of hypertension, said nutritional supplement comprising:
quercetin or a quercetin glycoside in an amount which provides a daily dosage of about 0.1 g to about 17.5 g per day;
a source of fat;
a source of carbohydrate; and
a source of protein, thereby providing a nutritional supplement which aids in preventing, delaying or slowing the onset or progression of hypertension.

19. The nutritional supplement of claim 20, further comprising a source of dietary fiber selected from the group consisting of soluble fiber, insoluble fiber, fermentable fiber, non-fermentable fiber and mixtures thereof.

20. A method of treating a subject, said method comprising:
measuring the subject's blood pressure,
comparing the thus measured blood pressure with a normal blood pressure for such a subject, and, if the measured blood pressure exceeds the normal blood pressure for such a subject, then diagnosing if the subject is suffering from or is at risk from suffering from hypertension,
if the subject is diagnosed as suffering from or at risk from suffering from hypertension, then directing the subject to ingest a nutritional supplement comprising quercetin, regularly, in amounts sufficient to treat hypertension or prehypertension in the subject, and
continuing to measure the subject's blood pressure at regular intervals to measure the efficacy of the ingestion of said nutritional supplement to treat the hypertension or reduce the risk of the subject suffering from hypertension.
